(19) United States
(12) Patent Application Publication
(10) Pub. No.: US 2010/0041703 A1
(43) Pub. Date: Feb. 18, 2010

(54) RAPID DISINTEGRATION MONOLAYER FILM FOR THE ORAL ADMINISTRATION OF ACTIVE SUBSTANCES

(75) Inventors: Michel Sournac, Toulouse (FR); Joël Bougaret, Francarville (FR); Isabel Ribeiro Dos Santos, Eaunes (FR)

Correspondence Address: BIRCH STEWART KOLASCH & BIRCH, PO BOX 747, FALLS CHURCH, VA, 22040-0747, US

(73) Assignee: Pierre Fabre Medicament, Boulogne-Billancourt (FR)

(21) Appl. No.: 12/529,118

(22) PCT Filed: Feb. 19, 2008
(86) PCT No.: PCT/EP2008/051972

§ 371 Date: Aug. 28, 2009

(30) Foreign Application Priority Data
Feb. 28, 2007 (FR) 0753562

(51) Int. Cl. A61K 31/454

(52) U.S. Cl. 514/323

(57) ABSTRACT

Rapid-disintegration monolayer film for the oral administration of active substances, comprising a water-soluble support containing at least one active substance, characterized in that it comprises a polymeric mixture of a hydrophilic film-forming agent formed from a copolymer of polyvinyl alcohol and of polyethylene glycol (PVA-PEG), of an active substance and of a hydrophilic gelling agent.

The present invention concerns rapid disintegration monolayer films intended for oral administering of a flavoured or cosmetic active pharmaceutical substance, their methods of preparation and their uses. When administered the film disintegrates in the mouth and releases the active substance.

Therefore the compositions subject of the invention are the ideal solution for ambulatory treatment. They allow rapid administering of an active substance without having recourse to a liquid to aid swallowing. The films such as described in the invention can be placed directly in the oral cavity e.g. on the palate or under the tongue where they disintegrate almost immediately.

Said property is therefore of particular advantage for the treatment of nausea, frequently travel sickness when the need for rapid administering is often combined with the impossible recourse to a liquid.

The compositions of the present invention also have an advantage for very young or elderly patients, who have trouble swallowing solid forms.

Rapid disintegration films must meet conditions which are often contradictory: they must firstly have a binding nature so that they can be shaped, and secondly they must have almost instant disintegrating capability. They must also have sufficient flexibility and resistance needed for example for their packaging in sachets, blisters or dispenser packs.

The soluble films currently used chiefly consist of hydrophilic polymers having film-forming properties, whose thickness and specific surface area can provide for rapid disintegration in contact with saliva.

These film-forming hydrophilic polymers are most often glucanes, natural gums or povidone derivatives. The combination of at least two polymers is generally desired to achieve a compromise between the main characteristics of the film i.e. flexibility, mechanical resistance and rate of disintegration.

For example in films containing cellulose polymers, the association of at least two water-soluble cellulose polymers is often necessary. Those containing the association of water-soluble cellulose polymers of high molecular weight (60000 to 150000 Da) and water-soluble cellulose polymers of low molecular weight are those most often encountered. The first type of polymer allows adjustment of the film's mechanical properties, whilst the second type of polymer allows adjustment of disintegration time.

International application WO 05/039499 for example describes a formulation for a rapid disintegration pharmaceutical or cosmetic film consisting of a combination of the type hydroxypropyl cellulose (Klucel JF®) and hydroxypropylmethyl cellulose (Methocel® grades E5, E50, E4M and SGA 16M). However, the films described in this application each contain several plasticizing agents (polyalcohols, sorbitan ester, citric acid esters . . . ) which are required to obtain flexible films but give rise to a complex formulation.

Also, polymers of glucane type such as pullulans are theoretically capable of forming rapid disintegration films on account of their high solubility, their rapid dissolution rate and their taste properties. However, their low molecular weight is a disadvantage and has the consequence of not facilitating the formation of films at concentrations lower than 20%; under these conditions, the viscosity of the mixtures is low and there is a risk that the mixtures may not be homogeneous when an insoluble constituent (active ingredient in dispersion) is added to the mixture. It is therefore necessary to associate other natural polymers to pullulans such as carrageenans or certain gums.

Application WO 03/030881 therefore describes a preparation in edible film form, preferably comprising a glucane and a water-soluble polymer at a ratio of between 40:1 and 0.5:1. This water-soluble polymer is characterized by a high molecular weight which makes it possible to increase the naturally low viscosity of the pullulans and to stabilize the mixture when it contains a suspension of active ingredient. Preferably, this polymer is a polysaccharide or a natural gum belonging to alginates, carrageenans, hydroxypropylmethyl cellulose, locust bean gum, guar gum, xanthan gum, dextrans, gum arabica, gellan gum either alone or in association. However the compositions described in this application contain anti-foam agents needed to obtain films devoid of air bubbles. It is also possible to associate cellulose polymers with natural polymers such as carrageenans or certain gums. For example, international application WO 04/105758 describes a ternary polymeric preparation, in the form of a quick-release edible film containing triprolidine intended for the treatment of sleep disorders and containing xanthan gum, hydroxypropylmethyl cellulose and carrageenan.

Natural gums (guar gum, locust bean gum, xanthan gum, alginates and carrageenans) or polyvinyl pyrrolidone are often considered to be stabilizing polymers, which need to be added either to cellulose derivatives or to glucanes to allow forming of the films. These conditions are not always satisfactory which leads to having recourse to the addition of a plasticizing compound such a polyoxyethylene sorbitan esters, fatty acid and glycerol esters, glycerol, fatty acid and propylene glycol esters, propylene glycol, dibutyl sebacate, triacetine.

Finally, numerous applications are characterized by the presence of a high number of constituents. For example, international application WO 03/030883 describes a preparation in edible film form based on the complex combination of hydroxypropylmethyl cellulose (Methocel E15®, polyvinyl pyrrolidone (PVP), corn starch (Pure Cote B792®), xanthan gum, surfactant (Cremophor EL®, plasticizer (propylene glycol) and preferably a taste masking agent.

International application WO 98/20862 describes an immediate-release film intended for the oral administering of medicinal or cosmetic active ingredients, consisting of water-soluble polymers, one or more polyalcohols, plasticizers, surfactants, flavouring and colouring agents. Preferably, the polymers are water-soluble cellulose derivatives such as hydroxypropylmethyl cellulose (HPMC), hydroxyethyl cellulose (HEC) and hydroxypropyl cellulose (HPC) either alone or in association. In the dry film, the concentration of cellulose polymer lies between 20 and 75 weight %. Other non-cellulose polymers may also be used such as polyvinyl pyrrolidone (PVP), carboxymethyl cellulose (CMC), polyvinyl alcohol (PVA), sodium alginate, polyethylene glycol (PEG), natural gums such as xanthan gum, tragacanth gum, guar gum, acacia gum, gum Arabica, water-dispersible polyacrylates such as polyacrylic acid, the methylmethacrylate polymer, carboxyvinyl copolymers.

International application WO 04/087084 describes a preparation in fast-release edible film form, consisting of cellulose film-forming polymers, more particularly a mixture of high viscosity polymers and low viscosity polymers in small concentration, which has an economic advantage. The cellulose polymers are of hydroxypropylmethyl cellulose (HPMC) type, preferably the Methocel® products series K or series E, and more specifically grades K4M, K100, K3, E50 and E4M. Here again, the presence of plasticizing agents in proportions ranging from 0.01% to 30% is required to obtain flexible non-brittle films.

International application WO04/045537 describes a complex preparation in the form of an edible film intended for the treatment of pharyngitis, consisting of pullulans, guar gum, pectins, xanthan gum, alginates, gelatin, starches, modified starches, maltodextrins, gluten, carboxymethyl cellulose, locust bean gum, carrageenans.

It was therefore necessary to develop a rapid-disintegration edible film having a simplified formulation compared with the complex compositions of the prior art, having equivalent or improved rheological properties (flexibility, mechanical resistance and disintegration rate).

The purpose of the present invention is to propose novel, rapid-disintegration monolayer films such as described below and illustrated in the examples, whose matrix essentially consists of two water-soluble polymers. The chief advantage of the films subject of the invention lies in the fact that they are devoid of plasticizer, whilst remaining sufficiently deformable without being brittle. Plasticizers are often hygroscopic, leading to difficult preservation and handling of the films obtained. Their use is therefore not always desirable. The films subject of the invention have the additional advantage of being able to accommodate a dispersed, insoluble, active substance in homogeneous manner. Finally, the films subject of the invention have the advantage of being obtained preferably by means of a mixing method conducted without an outside heating source, so as to preserve the fragile active substance they contain throughout the formation process. This last characteristics also has an economic advantage.

The present invention therefore concerns a rapid-disintegration monolayer film for the oral administering of active substances, comprising a water-soluble support containing at least one active substance, characterized in that it comprises a polymeric mixture of a hydrophilic film-forming agent consisting of a polyvinyl alcohol and polyethylene glycol (PVA-PEG) copolymer, an active substance and a hydrophilic gelling agent.

By rapid-disintegration film is meant flexible galenic forms having a thickness of less than 100 microns and whose disintegration, dissolution, dispersion or decomposition time in the oral cavity is less than one minute, advantageously less than 30 seconds, advantageously in the order of ten or so seconds. The active substance contained in these films can therefore be absorbed by passing of oral or sublingual mucosa or by swallowing.

By ambient temperature is meant a temperature of between 18° C. and 25° C., advantageously between 20° C. and 25° C.

The hydrophilic film-forming agent used in the invention is a grafted copolymer containing a film-forming fragment and a plasticizing fragment, the film-forming fragment being a polyvinyl alcohol (PVA) and the plasticizing fragment being polyethylene glycol (PEG).

In one particularly advantageous formulation the film-forming agent is Kollicoat IR® (marketed by BASF). Its molecular weight is around 45000 Da and it is easily soluble in water. The two parts of Kollicoat IR® contribute towards the mechanical properties of the film obtained. The PVA part imparts film-forming properties whilst the PEG part behaves as internal plasticizer. This type of film-forming agent is already known in the prior art, but it is usually utilized to coat tablets (WO 03/075896) since it allows low viscosity preparations to be obtained that are particularly appreciated for their film-forming properties.

However, this low viscosity at the concentrations used (at least 60 weight %) does not allow coating of a support to obtain a film without having recourse to the addition of a gelling agent. In unexpected manner, different associations between gelling, cellulose, water-soluble polymers and Kollicoat IR® have been made by the inventors without being able to obtain homogeneous mixtures promoting the stability of the film at the time of coating. For example, in order to increase the viscosity of a Kollicoat IR® solution, different grades of hydroxypropyl cellulose (HPC) of the type Blanose 7HF® or hydroxypropylmethyl cellulose (HPMC) or Methocel K15M® were used, but the mixtures obtained were heterogeneous and led to flocculation unsuitable for quality coating.

By coating is meant the step consisting of spreading the water-soluble support over a planar surface.

Advantageously, the gelling agent of the formulation is not a cellulose derivative. Advantageously, the gelling agent is a compound of the carrageenan family, in particular iota-carrageenan.

The gelling agent of particularly advantageous formulation is Gelcarin 379® (marketed by FMC Biopolymer), a compound of the iota-carrageenan family. The Applicant has found that it allows a homogeneous increase in the viscosity of the aqueous Kollicoat IR® solution without application of any heat, by producing a surfactant effect which promotes the stability of the mixture during coating. Gelcarin 379® remains dispersed and is not fully dissolved in the polymeric mixture, since it is not heated until the drying step. Advantageously, if carrageenans are used the weight proportion of film-forming agent to gelling agent lies between 1 and 10, advantageously between 3 and 8.

In formulations based on the association of Kollicoat and Gelcarin, the weight quantity of film-forming agent in the polymeric mixture represents 10% to 30% of the total weight, preferably 15% to 20%, and the weight quantity of gelling agent in the polymeric mixture represents 1% to 8% of the total weight, preferably 2% to 6%.

According to another variant of the invention, the gelling agent of the formulation may be gellan gum or xanthan gum.

If xanthan gum is used, the proportion by weight of film-forming agent to gelling agent lies between 10 and 200, advantageously between 25 and 100.

The quantity by weight of xanthan gum in the polymeric mixture lies between 0.1% to 0.5% of the total weight and advantageously lies between 0.2% and 0.45% of the total weight.

If gellan gum is used, the proportion by weight of film-forming agent to gelling agent lies between 100 and 400, advantageously between 150 and 300.

The quantity by weight of gellan gum in the polymeric mixture ranges from 0.01% to 0.5% of the total weight and advantageously from 0.02% to 0.2% of the total weight.

According to one advantageous characteristic, the homogeneous polymeric mixture is fully devoid of an additional plasticizer i.e. other than the one present in the film-forming polymer.

The homogeneous mixture may further contain additional components such as one or more surfactants. One preferred surfactant is polysorbate 80 (also called Tween 80).

Any active substance can be added to the film, whether in dispersed form or dissolved in the polymeric mixture. Amongst the active ingredients suitable for producing the composition of the invention, non-limiting mention may be made of those chosen from the group consisting of medicinal products, flavourings or food additives such as sweeteners.

The homogeneous mixture may additionally comprise additional components such as one or more surfactants and/or one or more fillers. One preferred surfactant is polysorbate 80 (also called Tween 80). The filler may be chosen from among mineral fillers conventionally used such as silica, talc or other silicate, titanium dioxide, but also from among some pharmaceutical thinners or lubricants such as magnesium, calcium or zinc stearates.

As non-limiting examples of medicinal active ingredients, mention may be made of anti-nausea agents e.g. domperidone, mequitazine, codein, loperamide hydrochloride, the combination between chlorhexidine digluconate and tetracaine, nicotine, oxybutinine and cetirizine. The active ingredient is advantageously domperidone.

The present invention also concerns a method to produce rapid-disintegration films according to the invention. Said method is characterized by the application of the following successive steps:

a) dispersion of the film constituents in water,

b) mixing and homogenization of the dispersion,

c) coating and drying of the mixture,

d) cutting the spools of film and packaging.

Advantageously, the dispersion in water of the film constituents is made by adding to more than one half of the quantity of water and in the following order: film-forming agent, surfactant, active ingredient, the remainder of water then the gelling agent.

According to one particular embodiment of the manufacturing method according to the invention, the film is obtained, without application of any external heat, by mixing a hydrophilic film-forming agent consisting of a polyvinyl alcohol and polyethylene glycol copolymer (PVA-PEG) having a viscosity of between 1 and 250 mPa·s, at ambient temperature, and an active substance with a gelling hydrophilic agent in the presence of water to obtain a homogeneous aqueous polymeric mixture, the proportion of film-forming agent to gelling agent being determined so as to impart a viscosity at ambient temperature of between 250 mPa·s and 15000 mPa·s to said homogeneous aqueous polymeric mixture, preferably between 1000 mPa·s and 10000 mPa·s, further preferably between 1500 mPa·s and 9000 mPa·s, before the forming of the film using a conventional coating and drying technique.

The films obtained after the drying step can be cut, either into individual units of size between 4 cm² and 10 cm², or they can be wound into rolls, or folded. In each of these situations, the films after coating may or may not be provided with a removable backing.

These films may be packaged in different manners, in individual sachets, pre-cut wafers, individual blister packs or dispenser packs.

The invention is illustrated below by the following non-limiting examples. Several rapid-disintegration films containing domperidone or another active ingredient were prepared from a homogeneous aqueous polymeric mixture of Kollicoat IR® and Gelcarin 379®. The proportions of the mixture and the physical properties of the films obtained after coating and drying are grouped together in Table 1 (laboratory tests) and Table 2 (pilot tests).

### EXAMPLE 1

Kollicoat IR® was added to 70% of the quantity of purified water under stirring. Stirring was continued until dissolution of Kollicoat IR®. Since the latter generates numerous bubbles, it can either be dissolved under a vacuum or the solution can be left to stand (as its viscosity is very low it will degas on its own). Tween 80 was incorporated in the solution under agitation under stirring, followed by the flavourings (condensed extract of liquorice and essential peppermint oil) and sweetener (acesulfame potassium). Stirring was continued until complete solubilisation of the powders. Domperidone was added under stirring until its dispersion in the mixture, then the remainder of the water (30%) was added. Gelcarin 379® was incorporated in the domperidone suspension under stirring to avoid the formation of aggregates. Aliquots of the mixture were then coated on a polyester support and dried using equipment of the type Lab Dryer Coater Mathis. The coated surfaces were cut using a manual press into units of 6 cm², then packaged manually in sealed sachets.

TABLE 1
Rapid-disintegration films containing a
Kollicoat IR/Gelcarin mixture, laboratory preparation.

| Film | | |
|---|---|---|
| 1 | 2 | 3 |

w/w % mixture

| | | | |
|---|---|---|---|
| Domperidone | 6 | 9.7 | 8.6 |
| Kollicoat IR | 15 | 17 | 15.8 |
| Gelcarin 379 | 5 | 3 | 2.7 |
| Tween 80 | 0.2 | 0.2 | 0.19 |
| Acesulfame potassium | 0.5 | 0.5 | 0.43 |
| Flavourings | 1.5 | 1.65 | 0.8 |
| Purified water | QS | QS | QS |
| Viscosity (mPa · s)¹ | 1540 | 1660 | 1980 |

Film properties

| | | | |
|---|---|---|---|
| Surface area (cm²) | 6 | 6 | 6 |
| Thickness (μm) | 65 | 40 | 40 |
| Gram weight (g/m²) | 87.3 | 60 | 63 |
| Water content (%) | 8.6 | 8.8 | 9 |
| Specific surface (cm²/g) | 220 | 334 | 334 |
| Tensile strength (N) | 6.5 | 12.9 | 16.50 |
| Elongation (%) | 3.2 | 3.1 | 4.3 |
| Disintegration time² (sec) | 12 | 7 | 15 |

¹Brookfield LVT3, 22° C., 20 rpm,
²50 ml water at 37° C.

### EXAMPLE 2

To produce batches of pilot size, the components were added to a vat in the following order: more than half of the quantity of water, Kollicoat IR®, sweeteners and flavourings (condensed extract of liquorice and essential peppermint oil), Tween 80 and Domperidone. The pre-mixture thus obtained was homogenized by successive passes through a die then re-added to the main vat. The remaining components (Gelcarin 379® and the residual quantity of water) were added to the premix and the whole was stirred. The final mixture was homogenized by three successive passes through a die then left to stand under reduced nitrogen pressure to allow good quality degassing before proceeding with the coating step. The mixture was transferred by means of a pump to a pilot coating line consisting of a coating station, three successive drying areas and a lamination station. The coating rate and width were respectively 0.85 m/min and 17 cm. A temperature gradient was set up firstly to avoid degradation of the active ingredient under the effect of heat, and secondly to target a residual quantity of water in the film compatible with its mechanical characteristics (film that is too dry will be too brittle and unsuitable for an handling). After a maturing time, the spool of film was cut and packaged using dedicated equipment.

TABLE 2
Rapid-disintegration films containing a
Kollicoat IR/Gelcarin mixture prepared on a pilot line.

| Film | | |
|---|---|---|
| 4 | 5 | 6 |

w/w % mixture

| | | | |
|---|---|---|---|
| Domperidone | 5.75 | 9.8 | 9.9 |
| Kollicoat IR | 15.6 | 18.2 | 18.2 |
| Gelcarin 379 | 5.2 | 3.1 | 3.1 |
| Tween 80 | 0.16 | 0.21 | 0.21 |
| Acesulfame potassium | 0.16 | 0.5 | 0.5 |
| Flavourings | 1.37 | 0.41 | 0.4 |
| Purified water | QS | QS | QS |
| Viscosity (mPa · s)¹ | 4400 | 7964 | 8496 |

Film properties

| | | | |
|---|---|---|---|
| Surface area (cm²) | 6 | 6 | 6 |
| Thickness (μm) | 65 | 46 | 45 |
| Gram weight (g/m²) | 88.4 | 59 | 58 |
| Water content (%) | 9.6 | 7.3 | 7.8 |
| Tensile strength (N) | 7.5 | 11.1 | 9.4 |
| Elongation (%) | 5.8 | 3.85 | 4 |
| Disintegration time² (sec) | 21.5 | 7 | 7 |
| Disintegration time³ (sec) | 21.6 | 10 | 10 |

¹Brookfield LV4, 25° C., 50 rpm,
²50 ml water at 37° C., 3Sotax, 800 ml water at 37° C.

### EXAMPLE 3

Other rapid-disintegration films were prepared from a homogeneous aqueous polymeric mixture of Kollicoat IR® and gellan gum or xanthan gum following the method of Example 1. The results are grouped together in Table 3.

TABLE 3
Fast-disintegration films containing a mixture
of Kollicoat IR/gellan gum (Kelcogel) or xanthan gum
(Kelgum) and containing domperidone.

Film

| | |
|---|---|
| 7 | 8 |

w/w % mixture

| | | |
|---|---|---|
| Domperidone | 8.2 | 9.8 |
| Kollicoat IR | 18.2 | 18.2 |
| Kelgum | 0.4 | |
| Kelcogel LT 100 | | 0.075 |
| Tween 80 | 0.18 | 0.17 |
| Purified water | QS | QS |
| Viscosity (mPa · s)¹ | 1900 | 2400 |

Film properties

| | | |
|---|---|---|
| Surface area (cm²) | 6 | 6 |
| Gram weight (g/m²) | 57.09 | 60.88 |
| Water content (%) | 10 | 10.24 |

¹Brookfield LV4, Spindle 2, 22.5° C., 10 rpm

### EXAMPLE 4

A variety of active ingredients was able to be integrated into the films of the invention. The compositions of the films obtained following the operating mode of Example 1 previously described are given in Table 4.

TABLE 4
Rapid-disintegration films containing various
active ingredients.

| | Film | | | | |
|---|---|---|---|---|---|
| Composition (mg) | 9 | 10 | 11 | 12 | 13 |
| Chlorhexidine digluconate | 2.53 | | | | |
| Tetracaine | 0.19 | | | | |
| Mequitazine | | 10 | | | |
| Caffein | | | 10 | | |
| Loperamide HCl | | | | 2 | |
| Codein Phosphate hemihydrate | | | | | 18.68 |
| Kollicoat IR | 21.88 | 18.49 | 18.49 | 25.32 | 17.73 |
| Iota-carrageenan | 1.67 | 3.12 | 3.14 | 4.30 | 2.99 |
| Polysorbate 80 | 0.25 | 0.21 | 0.21 | 0.22 | 0.33 |
| Acesulfame potassium | 0.46 | 0.51 | 0.51 | 0.51 | 0.63 |
| Ammonium glycyrrhizinate | 0.19 | 0.22 | 0.21 | 0.21 | 0.27 |
| Flavourings | — | 0.21 | 0.20 | 0.20 | 0.33 |
| Purified water | QS | QS | QS | QS | QS |
| Total | 30.0 | 36.0 | 36.0 | 36.0 | 45.0 |

### EXAMPLE 5

Another rapid-disintegration film which gave particularly satisfactory results was prepared from a homogeneous, aqueous polymeric mixture of a PVA-PEG copolymer (Kollicoat IR®) and an iota-carrageenan (Gelcarine 379®) following the method described previously.

The results are given in Table 5.

TABLE 5
Unit composition of a rapid-disintegration
film containing Domperidone.

Film 14

| End product | mg | w/w % |
|---|---|---|
| Domperidone | 10.0 | 30.3 |
| Kollicoat IR | 16.85 | 51.0 |
| Gelcarine 379 | 2.73 | 8.3 |
| Tween 80 | 0.20 | 0.6 |
| Acesulfame potassium | 0.50 | 1.51 |
| Ammonium glycyrrhizate | 0.20 | 0.6 |
| Extract of liquorice | 0.04 | 0.12 |
| Peppermint | 1.0 | 3.03 |

| | |
|---|---|
| Water | QS |

| | | |
|---|---|---|
| Total | 33.0 | 100.0 |

1. Rapid disintegration monolayer film for the oral administering of active substances, comprising a water-soluble support containing at least one active substance, characterized in that it comprises a polymeric mixture of a hydrophilic film-forming agent consisting of a copolymer of polyvinyl alcohol and polyethylene glycol (PVA-PEG), an active substance and a hydrophilic gelling agent.

2. Film according to any of the preceding claims, characterized in that the hydrophilic gelling agent is chosen from the list comprising carrageenans preferably iota-carrageenan, gellan gum and xanthan gum.

3. Film according to any of the preceding claims, characterized in that the polymeric mixture is devoid of an additional plasticizer.

4. Film according to claim 2, characterized in that the proportion by weight of film-forming agent to hydrophilic gelling agent lies between 1 and 10 when the latter belongs to the carrageenan family, preferably between 3 and 8.

5. Film according to claim 4, characterized in that the quantity by weight of film-forming agent in the polymeric mixture represents 10% to 30% of the total weight, preferably 13% to 20%, and the quantity by weight of hydrophilic gelling agent in the polymeric mixture represents 1% to 8% of the total weight, preferably 2% to 6%.

6. Film according to claim 2, characterized in that when the gelling agent is an xanthan gum, the proportion by weight of film-forming agent to hydrophilic gelling agent lies between 10 and 200, preferably between 25 and 100.

7. Film according to claim 6, characterized in that the quantity by weight of xanthan gum in the polymeric mixture lies between 0.1% and 0.5% of the total weight, preferably between 0.2% and 0.45% of the total weight.

8. Film according to claim 2, characterized in that when the gelling agent is a gellan gum, the proportion by weight of film-forming agent to hydrophilic gelling agent lies between 100 and 400, preferably between 150 and 300.

9. Film according to claim 8, characterized in that quantity by weight of hydrophilic gelling agent in the polymeric mixture lies between 0.01% and 0.5% of the total weight, preferably between 0.02% and 0.2% of the total weight.

10. Film according to any of the preceding claims, characterized in that the active substance is chosen from the group comprising domperidone, mequitazine, codein, loperamide hydrochloride, the combination between chlorhexidine digluconate and tetracaine, nicotine, oxybutinine and cetirizine, and is preferably domperidone.

11. Film according to any of the preceding claims, characterized in that it further contains one or more fillers and/or one or more surfactants.

12. Film according to any of the preceding claims, characterized in that it contains polysorbate 80 as surfactant.

13. Method to produce a film according to any of the preceding claims, characterized by conducting the following successive steps:
a) dispersing the film constituents in water,
b) mixing and homogenizing the dispersion,
c) coating and drying the mixture.

14. Method according to claim 13, characterized in that the dispersion in water of the film constituents is obtained by adding to more than one half of the quantity of water and in the following order: film-forming agent, surfactant, active ingredient, remainder of the water, then the hydrophilic gelling agent.

15. Method according to either of claims 12 and 13, characterized in that the film is obtained without applying any external heat by mixing a hydrophilic film-forming agent, consisting of a copolymer of polyvinyl alcohol and polyethylene glycol (PVA-PEG) of viscosity between 1 and 250 mPa·s at ambient temperature, an active substance and a hydrophilic gelling agent in the presence of water to obtain a homogeneous aqueous polymeric mixture, the proportion of film-forming agent to gelling agent being determined so as to impart to said homogeneous aqueous polymeric mixture a viscosity at ambient temperature of between 250 mPa·s and 15000 mPa·s, preferably between 1000 mPa·s and 10000 mPa·s, further preferably between 1500 mPa·s and 9000 mPa·s before forming of the film using a conventional coating and drying technique.

16. Film according to any of claims 1 to 12 as medicinal product.
